(19) United States
(12) Patent Application Publication
(10) Pub. No.: US 2017/0136180 A1
(43) Pub. Date: May 18, 2017

(54) Stretchable, Robust and Biocompatible Hydrogel Electronics and Devices

(71) Applicant: Massachusetts Institute of Technology, Cambridge, MA (US)

(72) Inventors: Xuanhe Zhao, Cambridge, MA (US); Shaoting Lin, Cambridge, MA (US); Hyunwoo Yuk, Cambridge, MA (US); Teng Zhang, Cambridge, MA (US)

(21) Appl. No.: 15/274,218

(22) Filed: Sep. 23, 2016

Related U.S. Application Data
(60) Provisional application No. 62/232,548, filed on Sep. 25, 2015.

(51) Int. Cl. A61M 5/172; A61K 9/70; A61K 9/06; A61M 35/00; A61K 9/00

(52) U.S. Cl. CPC A61M 5/1723; A61M 35/00; A61K 9/0014; A61K 9/0053; A61K 9/06; A61K 9/7007; A61K 9/0009; A61M 2230/005; A61M 2230/50

(57) ABSTRACT

A tough biocompatible hydrogel having one or more drug delivery components, deformable conductors, and/or rigid electronic components incorporated therein in such a way that robust interfaces are formed between the hydrogel and the various components. The resulting hydrogel device provides a highly deformable hydrogel composite in which the reliability and functionality of the incorporated components are maintained even under states of large deformation, and from which one or more drugs can be delivered in a controlled and sustained manner regardless of the state of deformation.

### RELATED APPLICATION(S)

This application claims the benefit of U.S. Provisional Application No. 62/232,548, filed on Sep. 25, 2015, the entire teaching of which is incorporated herein by reference.

This invention was made with Government support under Grant No. N00014-14-1-0619 awarded by the Office of Naval Research and under Grant No. CMMI-1532136 awarded by the National Science Foundation. The Government has certain rights in the invention.

### FIELD OF THE INVENTION

The present invention relates to a tough biocompatible hydrogel having one or more components, such as drug delivery components, stretchable or otherwise deformable conductors, and rigid electronic components, incorporated therein. Robust interfaces are formed between the hydrogel and the various components to provide a mechanically strong and highly stretchable hydrogel composite in which the reliability and functionality of the components are maintained even under states of large deformation.

### BACKGROUND OF THE INVENTION

Hydrogels are hydrophilic polymeric materials capable of holding large amounts of water in their three-dimensional networks. They are typically made using natural polymers (e.g., collagen and alginate) or synthetic polymers (e.g., poly(vinyl alcohol) (PVA) and poly(acrylic acid) (PAA)). Depending on the nature of the hydrogel network, they can be categorized as either “physical” hydrogels, which means that the network formation is reversible, or “chemical” hydrogels, which means that the network formation is irreversible and is formed by covalent cross-links. Due to their high water content, porosity, and soft consistency, hydrogels closely resemble natural living tissue. These properties, along with their generally good biocompatibility and ease of fabrication, make hydrogels desirable for use in a number of biomedical applications. In particular, hydrogels are widely used in forming contact lenses, wound dressings, drug delivery devices, and hygiene products, as well as in tissue engineering.

However, while hydrogels possess many beneficial properties, they also have some limitations—mainly poor mechanical properties and week hydrogel-solid interfaces. For example, hydrogels possess low tensile strength which limits their use in applications requiring load-bearing. In such load-bearing applications, hydrogels are typically unable to maintain their shape and function in the long-term. Further, tissue engineering using hydrogels has generally resulted in hydrogel tissues having significantly poorer mechanical strength than the real tissue. In particular, most hydrogels are brittle and possess very low stretchability. For example, typical fracture energies of hydrogels are about 10 J m⁻² as compared with ˜1,000 J m⁻² for cartilage and ˜10,000 J m⁻² for natural rubbers. Moreover, formation of week hydrogel-solid interfaces results in a failure to integrate the soft hydrogel and rigid components with adequate functionality and reliability. Further, in drug delivery applications, it may be problematic to load and effectively deliver certain drugs from hydrogels. In particular, in the case of hydrophobic drugs, the high water content and high porosity of most hydrogels can result in rapid drug release rather than a desired slower and sustained release of the drug.

Various attempts have been made to address these limitations. However, in view of the great potential that hydrogels possess for use in various applications, further improvements are still needed.

### SUMMARY OF THE INVENTION

Embodiments of the present invention provide hydrogel-based devices that are mechanically robust, highly stretchable, biocompatible, and capable of programmable delivery and sustained release of drugs, and methods of their production. In particular, the hydrogel-based devices incorporate various components in such a way that drug delivery and functionality of integrated components are maintained in both undeformed and highly deformed states.

According to one aspect, the present invention provides a therapeutic agent delivery system comprising a stretchable hydrogel, at least one therapeutic agent reservoir disposed within the stretchable hydrogel, and at least one electronic sensor disposed within or on the stretchable hydrogel and in communication with the at least one therapeutic agent reservoir, wherein the therapeutic agent contained within the at least one therapeutic agent reservoir is delivered through the stretchable hydrogel in response to one or more conditions detected by the at least one electronic sensor component.

According to another aspect, the present invention provides a transdermal therapeutic agent delivery system comprising a stretchable hydrogel sheet, at least one therapeutic agent reservoir disposed within the stretchable hydrogel sheet, and at least one electronic sensor disposed within or on the stretchable hydrogel and in communication with the at least one therapeutic agent reservoir, wherein the delivery of therapeutic agent contained within the at least one therapeutic agent reservoir is regulated based upon one or more conditions detected by the at least one electronic sensor.

Embodiments according to these aspects can include one or more of the following features. The at least one electronic sensor is in communication with the at least one therapeutic agent reservoir via one or more flexible conductors disposed within the stretchable hydrogel. The transdermal therapeutic agent delivery system can further comprise a therapeutically effective dosage of the therapeutic agent. The stretchable hydrogel sheet can be configured to adhere and conform to a surface on which it is placed. The transdermal therapeutic agent delivery system can further comprise at least one channel in connection with the at least one reservoir for delivering one or more therapeutic agents into the at least one reservoir. The at least one electronic sensor can be a temperature sensor, and delivery of therapeutic agent contained within the at least one therapeutic agent reservoir can be regulated based upon temperature. The stretchable hydrogel can have a stiffness of about 10 to 100 kilopascals. The stretchable hydrogel can be in the form of an ingestible capsule or tablet. The stretchable hydrogel can be in the form of an implant.

According to another aspect, the present invention provides a method for transdermal delivery of one or more therapeutic agents comprising providing a stretchable hydrogel sheet, the stretchable hydrogel sheet having at least one therapeutic agent reservoir disposed therein, at least one fluid delivery channel in connection with the at least one therapeutic agent reservoir, and at least one electronic sensor disposed within or on the stretchable hydrogel and in communication with the at least one therapeutic agent reservoir; disposing the stretchable hydrogel sheet on a surface of a subject's skin; delivering at least one therapeutic agent to the at least one therapeutic agent reservoir via the at least one fluid delivery channel; sensing one or more conditions of the subject using the electronic sensor; and automatically regulating delivery of the at least one therapeutic agent contained within the at least one therapeutic agent reservoir through the hydrogel sheet and to the subject based upon the one or more conditions.

Embodiments according to this aspect can include one or more of the following features. The method can further comprise, prior to disposing the stretchable hydrogel sheet on the surface of the subject's skin, stretching the hydrogel sheet and disposing the stretched hydrogel sheet on the surface of the subject's skin. The hydrogel sheet can be disposed on a wounded or burned surface of the subject's skin. At least one condition of the subject can be temperature, and the therapeutic agent can be delivered to the subject upon an increase in temperature above a threshold level.

According to another aspect, the present invention provides a method for delivery of one or more therapeutic agents to a subject comprising providing a stretchable hydrogel, the stretchable hydrogel having at least one therapeutic agent reservoir disposed therein, at least one therapeutic agent contained within the at least one therapeutic agent reservoir, and at least one electronic sensor disposed within or on the stretchable hydrogel and in communication with the at least one therapeutic agent reservoir; delivering the stretchable hydrogel to a subject internally; sensing one or more conditions of the subject using the electronic sensor; and automatically regulating delivery of the at least one therapeutic agent contained within the at least one therapeutic agent reservoir through the hydrogel and to the subject based upon the one or more conditions.

Embodiments according to this aspect can include one or more of the following features. Delivering can comprise implanting. Delivering can comprise ingesting.

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are included to provide a further understanding of the invention, and are incorporated in and constitute a part of this specification. The components in the drawings are not necessarily to scale, emphasis instead being placed upon clearly illustrating the principles of the present invention. The drawings illustrate embodiments of the invention and, together with the description, serve to explain the principals of the invention.

FIGS. 1A-B schematically illustrate a deformable hydrogel electronic device according to an embodiment of the present invention, with FIG. 1A depicting an undeformed state and FIG. 1B depicting a stretched state.

FIGS. 2A-D illustrate a hydrogel conductor device in which wavy titanium wires are encapsulated in a tough hydrogel matrix according to an embodiment of the present invention, with FIG. 2A schematically illustrating both undeformed and deformed states, FIG. 2B depicting a simulation that demonstrates the maximum principal strain of the device, FIG. 2C graphically showing λₘₐₓ as a function of A/L, in both silanized wire surfaces and pristine wire surfaces, and FIG. 2D graphically showing resistance as a function of stretch over multiple cycles.

FIG. 3A schematically illustrates the integration of rigid PDMS chips on the surface of a tough hydrogel matrix via a glass slide adhesion layer according to an embodiment of the present invention, with FIG. 3B demonstrating no debonding of a chemically anchored PDMS chip even when pulled by a tweezer, FIG. 3C demonstrating debonding of a physically attached PDMS chip, FIG. 3D graphically showing G/μL as functions of λ and L/S for chemically and physically anchored interfaces, FIG. 3E demonstrating no debonding of a chemically anchored PDMS chip under high stretch, FIG. 3F illustrating a sheet of hydrogel with multiple patterned chips therein as attached a body part that causes deformation of the sheet, and FIG. 3G illustrating a hydrogel electronic device encapsulating an array of LED lights connected by stretchable silanized titanium wire.

FIGS. 4A-F illustrate the integration of drug-delivery channels in a hydrogel matrix according to an embodiment of the present invention, with FIG. 4A schematically illustrating diffusion of a drug in both deformed and stretched states, FIG. 4B showing experimental snapshots of drug diffusion in an undeformed hydrogel, FIG. 4C showing experimental snapshots of drug diffusion in a deformed hydrogel, FIG. 4D graphically showing normalized one-dimensional diffusion of a mock drug from inside an undeformed hydrogel channel, FIG. 4E graphically showing normalized one-dimensional diffusion of mock drug inside a deformed (λ=1.6) hydrogel channel, and FIG. 4F showing experimental snapshots of the diffusion of multiple mock drugs in a hydrogel matrix under no stretch and under high stretch.

FIG. 5A-F illustrate a smart wound dressing according to an embodiment of the present invention, with FIG. 5A schematically illustrating temperature sensors patterned in a 3 by 3 matrix with a drug-delivery reservoir next to each, FIG. 5B graphically showing the temperatures at different locations on the skin measured via each of the temperature sensors over time, and Figs. C-F illustrating injection of mock drugs into a reservoir corresponding to a temperature sensor that has detected increase of temperature over a certain level, and subsequent sustained release of the injected drugs over time.

### DETAILED DESCRIPTION

The following definitions are useful for interpreting terms applied to features of the embodiments disclosed herein, and are meant only to define elements within the disclosure.

As used herein, the term “deformed” refers to any state in which the device is exposed to external force that results in a change in the natural state of the device and can include, for example, stretching, bending, and twisting. As such, when the device is referred to as being deformed, it can include any of these possible forms of deformation as well as any other conventional types of deformation that are not specifically mentioned herein.

As used herein, the terms “functional” and “reliable”, when referring to the deformable hydrogel devices and components as “maintaining their functionality and reliability”, generally means that the overall operation of the hydrogel device remains the same or substantially the same regardless of the state of deformation. In particular, deformation of the hydrogel device does not cause the components to become disengaged or disconnected from the hydrogel. Further, deformation of the hydrogel device does not cause the components (i.e., the drug delivery components, the rigid electronic components, and the flexible conductor components) to cease operation or to operate differently or substantially differently than the components operate when the hydrogel device is not subjected to deformation. A substantial difference in functionality or reliability of the components would be a difference that results in the hydrogel device not functioning as needed or as intended (e.g., failure of the device to deliver a therapeutically effective dose of one or more drugs as intended, failure of one or more sensors to properly monitor one or more conditions, failure of delivery of one or more drugs upon one or more conditions being sensed, failure of the sensors to transmit measured conditions, etc.).

The present invention generally provides novel hydrogel devices that are tough and deformable, and which can incorporate one or more rigid components, stretchable components, and/or drug delivery components in such a way that the hydrogel device maintains its functionality and reliability in both undeformed and highly deformed states. In particular embodiments, the present invention provides methods for integrating flexible conductors, rigid electronic components, and drug-delivery channels and reservoirs into and/or onto biocompatible and tough hydrogel matrices that contain significant amounts of water (e.g., 70˜95 wt %) by using a combination of novel tough hydrogels and fabrication methods. The resultant hydrogel-based devices are mechanically robust, highly stretchable, biocompatible, and capable of multiple functions.

Reference will now be made in detail to embodiments of the present invention, examples of which are illustrated in the accompanying drawings. Wherever possible, the same reference numbers are used in the drawings and the description to refer to the same or like parts.

According to one aspect, the present invention combines a tough hydrogel matrix and robust hydrogel-solid interfaces (i.e., interfaces between the hydrogel and the integrated rigid and stretchable components) to provide deformable hydrogel devices that are functional and reliable even under states of large deformation.

In order to design a tough hydrogel matrix, long-chain polymer networks that are highly deformable are combined with one or more additional components that are capable of dissipating significant mechanical energy under deformation. These two components (i.e., long chain networks and dissipative components) are interpenetrated with each other after curing in such a way that they work synergistically, with the long chain networks functioning to maintain the integrity of the material, and the dissipative components (e.g. ionic crosslinks, fiber filler, etc.) providing mechanical energy dissipation when the whole polymer network undergoes deformation.

In particular, when the whole polymer network sustains deformation, the long chain network stretches, while the dissipative network breaks to release mechanical energy, which retards the propagation of cracks in the network. The mechanical dissipation is generally triggered once loading deformation reaches a certain amount, which largely upon the extension limit of the specific dissipative network and will vary for different types of dissipative networks. According to preferred embodiments, the dissipative network is reversibly dissipative (e.g., contains reversible crosslinks) such that the damaged dissipative network gradually reforms to at least partially “heal” the strength of the material. Thus, the dissipative network acts to “unzip” (i.e., break) and “zip” (i.e., heal) the network upon deformation. Both the stretchability of the long chain networks and the reversible damage of the dissipative networks work synergistically to enhance the toughness of the whole material by orders of magnitude.

The long-chain polymer networks that are highly deformable can be selected from any known deformable long-chain polymer networks. Since the tough hydrogels can be used in a wide variety of biomedical applications, the polymers used in the present invention are preferably biocompatible (although for non-biomedical applications it would not be necessary to utilize only biocompatible polymer materials). In general, molecular weight determines the stretchability of the network, with larger molecular weights typically resulting in higher stretchability. As such, polymers having a high molecular weight associated with higher stretchability are typically used to provide the long-chain polymer networks. Some examples of highly deformable long-chain polymer networks include, but are not limited to, polyacrylamide (PAAm), polyethylene glycol (PEG) and N,Ndimethylacrylamide (DMMA). According to an exemplary embodiment, these long-chain polymer networks are covalently crosslinked. For example, as shown in Table 1, two exemplary long-chain polymer networks include polyacrylamide (PAAm) covalently crosslinked by N,N-methylenebisacrylamide (BIS), and polyethylene glycol (PEG) covalently crosslinked by diacrylate (DA).

TABLE 1
Compositions of a Set of Tough Hydrogels
With Long-Chain and Dissipative Networks

| | | Dissipative networks | | |
|---|---|---|---|---|
| | | Alginate | HA | Chitosan |
| Long- | PAAm | 12 wt. % AAm, | 18 wt. % AAm, | 24 wt. % AAm, |
| chain | | 0.017 wt. % MBAA, | 0.026 wt. % MBAA, | 0.034 wt. % MBAA, |
| networks | | 2 wt. % alginate, | 2 wt. % HA, | 2 wt. % chitosan, |
| | | 200 μL of CaSO₄ | 60 μL of iron (III) | 60 μL of TPP |
| | | (1M) per 10 mL | chloride (0.05M) per | (0.05M) per 10 mL |
| | | precursor | 10 mL precursor | precursor |
| | PEGDA | 20 wt. % PEGDA, | 20 wt. % PEGDA, | 20 wt. % PEGDA, |
| | | 2.5 wt. % alginate, | 2 wt. % HA, | 2 wt. % Chitosan, |
| | | 200 μL of CaSO₄ | 60 μL of iron (III) | 60 μL of TPP |
| | | (1M) per 10 mL | chloride (0.05M) per | (0.05M) per 10 mL |
| | | precursor | 10 mL precursor | precursor |

The one or more additional components that are capable of dissipating significant mechanical energy under deformation can, likewise, be any such known components. For example, according to various embodiments, mechanical dissipation is incorporated in the present materials by including ionic crosslinks, pull-out fibers, and/or transformation domain(s) in the polymer chains. In some embodiments, the additional dissipative components may comprise polymer networks. According to particularly preferable embodiments, the dissipative material is reversibly dissipative, which means that the material can reform after damage to at least partially heal the strength of the material. Some examples of the dissipative components include, but are not limited to, alginate, hyaluronan and chitosan, which are all biocompatible materials. Such materials preferably contain reversible crosslinks, which enables the kinetics of the zipping and unzipping process of the dissipative materials, inhibits the propagation of cracks, and enhances anti-fatigue performance of the material. Thus, the reversible crosslinks not only provide energy dissipation from the breakage of the crosslinks, but also ensures the anti-fatigue performance due to reforming (i.e., healing) of the damaged crosslinks. According to the exemplary embodiments shown in Table 1, the dissipative components can include, alginate reversibly crosslinked by calcium sulfate, hyaluronan reversibly crosslinked by iron (III) chloride, and chitosan reversibly crosslinked by sodium tripolyphosphate. Of course, other combinations are possible and could be determined by one skilled in the art.

According to embodiments of the present invention, tough and biocompatible hydrogels are formed using individual polymer networks of PAAm, PEG, alginate, hyaluronan or chitosan, as well as PAAm-alginate and PEG-alginate. Among the materials presented in Table 1, biocompatible PAAm-alginate hydrogel was determined to provide the highest stretchability (˜21 times) and fracture toughness (˜9,000 Jm⁻²). As such, additional studies were performed using PAAm-alginate as the hydrogel matrix for stretchable electronics and devices. These additional studies are described herein in further detail. However, it is to be understood that the present invention is not limited to this particular material (PAAm-alginate), and that additional tough hydrogels listed in Table 1, as well as other components possessing the properties outlined herein, can also suitably be used in the present invention. For example, according to various embodiments of the present invention, a hydrogel matrix that combines long-chain polymer networks that are highly deformable with one or more additional components that are capable of dissipating significant mechanical energy under deformation would be included in the present invention provided that the resulting hydrogel matrix provides a stretchability of at least about 5 times, more preferably at least 8 times, and/or a fracture toughness of at least about 1000 Jm⁻².

According to some embodiments, PEG-alginate and PEG-hyaluronan hydrogel matrices, while possibly possessing less stretchability and toughness than PAAm-alginate, have been determined to beneficially allow for encapsulation of viable cells. As such, these materials can be particularly useful in forming hydrogel devices that incorporate viable cells (i.e., for delivery of viable cells to a subject).

In order to provide tough, deformable hydrogel devices that contain solid components (e.g., rigid and stretchable components) which are functional and reliable even under states of large deformation, robust bonds are formed between the tough hydrogels and the solid components. These robust bonds are formed by chemically anchoring the long-chain network of the polymer in the tough hydrogels onto the surfaces of the solid components. This chemical anchoring enables stable bonding of hydrogels having relatively high intrinsic adhesion energy and large enhanced surface toughness from the dissipative networks contained therein. Preferably, robust bonding of tough hydrogels onto various solids is achieved by surface functionalization (i.e., modifying the surface using physical, chemical or biological mechanisms) of the component surface(s). In preferred embodiments, functional silanes are grafted onto the solid component surfaces followed by activation of surface oxides (e.g., using either oxygen plasma or UV-ozone treatment).

According to one exemplary embodiment, functional silane 3-(trimethoxysilyl)propyl methacrylate (TMSPMA; Sigma, 440159) was used to treat the surface of a rigid substrate. In particular, the rigid substrate was first cleaned with acetone, ethanol and deionized water, followed by treatment with oxygen plasma for about 5 min. After oxygen plasma treatment, the solid surface was covered with an aqueous silane solution (2-5 wt. % TMSPMA in deionized water with pH 3.5). After an hour of incubation at room temperature, the rigid surface was thoroughly cleaned with ethanol and completely dried. The prepared substrate was then used to provide robust bonding with a tough hydrogel by curing a polyacrylamide-alginate tough hydrogel precursor solution onto the functionalized rigid solid substrate. It is noted that a similar procedure can be carried out on various other types of tough hydrogels and solid substrate materials by modifying the appropriate hydrogel precursor solutions and functional materials (e.g., silanes) for each solid substrate.

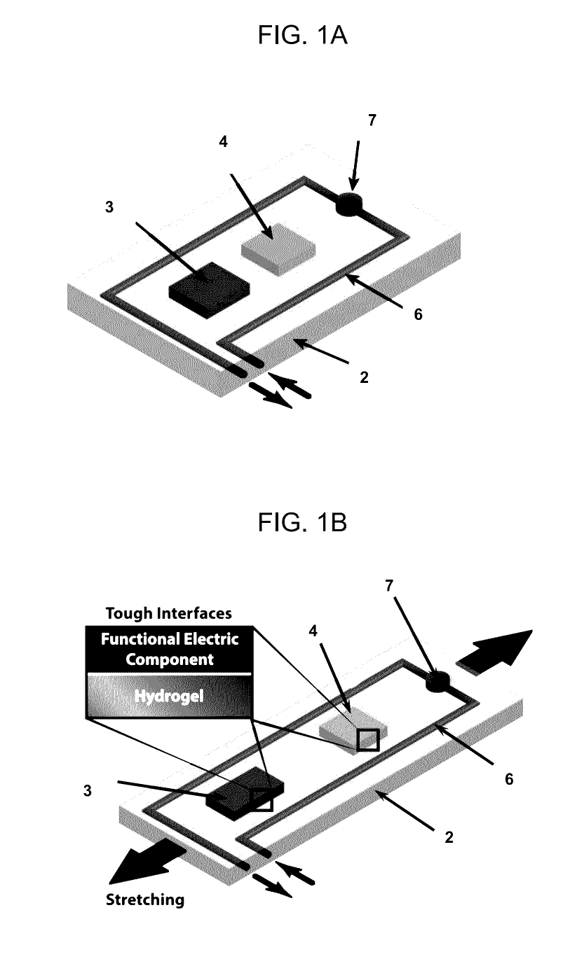

As such, the present invention provides a method to make deformable, robust, and biocompatible hydrogel electronic devices which can include the following three general components: one or more flexible components 3 embedded inside the hydrogel 2, one or more rigid components 4 encapsulated inside of or disposed on a surface of hydrogel 2, and one or more drugs 5 disposed inside the hydrogel 2 so as to provide a desired delivery of the drugs 5. This is depicted, for example, in FIGS. 1A-B. In particular, as shown in FIG. 1A, one or more functional electronic components, which can be either flexible or rigid components 3, 4 (e.g., conductors, microchips, transducers, resistors, and capacitors) are embedded inside of or are attached to a surface of the hydrogel 2. Drug-delivery channels 6 and reservoirs 7 are patterned in the hydrogel 2 matrix. These channels 6 and reservoirs 7 are configured and arranged within the hydrogel 2 so as to diffuse one or more drugs 5 out of the hydrogel electronic device 1 to provide programmable and sustained release of the one or more drugs 5. As depicted in FIG. 1B, as the hydrogel electronic device 1 is deformed (here, by stretching) the flexible electronic components 3 deform together with the device 1. On the other hand, the rigid components 4 maintain their undeformed shapes, which requires robust interfaces between the rigid components 4 and the hydrogel 2 matrix.

According to embodiments of the present invention, the channels and reservoirs are patterned into the hydrogel material. In particular, according to an exemplary embodiment, patterned channels and reservoirs are formed by first pouring a precursor hydrogel solution (e.g., a PAAm-alginate precursor solution) into a patterned mold. Any conventional type of mold may be used, but an acrylic mold was used in the present examples. Thereafter, the precursor solution is cured. For example, a PAAm-alginate precursor solution may be cured by subjection to ultraviolet light for an extended period of time, (e.g., about 60 minutes with approximately 8 W power and about 254 nm wavelength). Suitable durations, power levels and wavelengths for the cure can be determined by one skilled in the art in light of the material being cured. After curing the precursor solution fully, a heated solution (e.g., a heated gelatin solution such as Knox at about 70° C.) is infused into the hydrogel to form reservoirs and channels. In particular, the heated gelatin solution may be infused into reservoirs with the desired diameter (e.g., a diameter of about 8 mm) and plastic tubes (e.g., McMaster, Outside diameter: 1.52 mm; wall thickness: 0.51 mm) to form the channels inside the cured hydrogel material. The hydrogel material with infused gelatin is then cooled (e.g., by placing in a refrigerator at about 5° C. for about 5 min). In embodiments utilizing temperature sensors in the hydrogel, at this point temperature sensors (e.g., 24PetWatch) can be placed near each of the reservoirs. The resultant material is again covered with the precursor solution so as to encapsulate both the solidified gelatin and micro sensors. The sample is further cured, for example, at about room temperature for about 30 min. After this second curing, the entire hydrogel material is heated to liquefy the solidified gelatin (e.g., by placing in an oven at about 70° C. for 10 minutes). Liquefied gelatin is then washed away from the hydrogel matrix by flowing a fluid (e.g., deionized water) through the channels and reservoirs as many times as necessary.

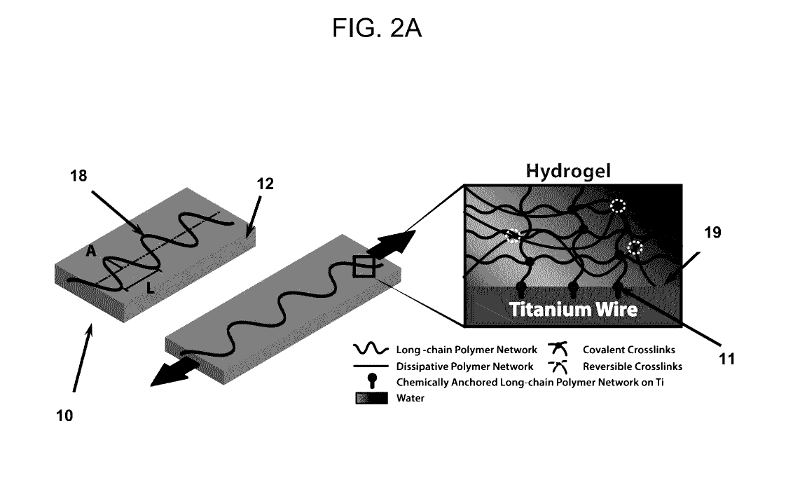

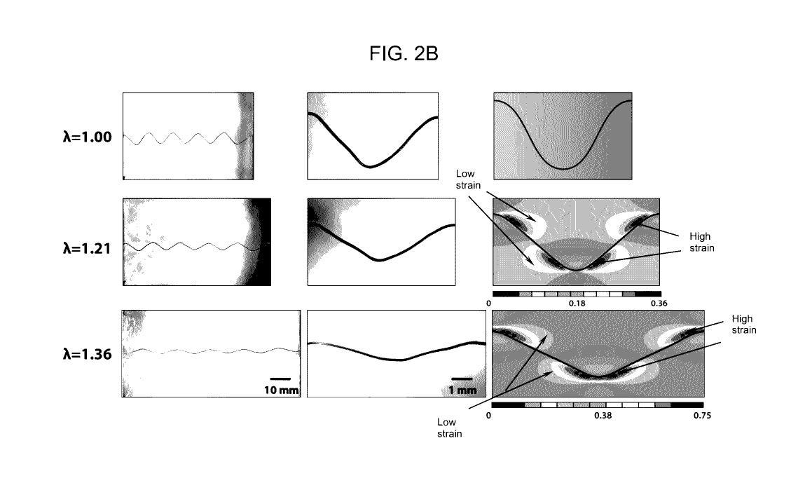

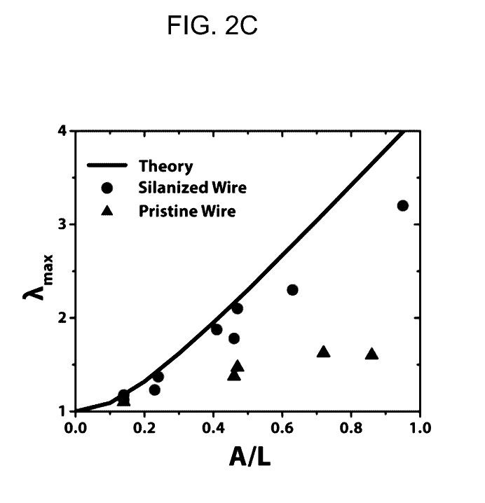

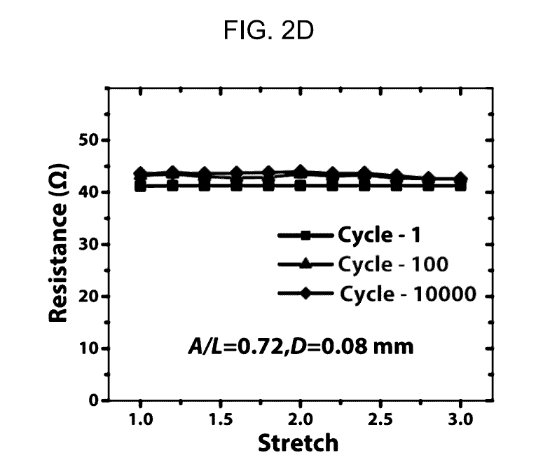

According to some embodiments, one or more flexible conductors are encapsulated within the hydrogel matrix material. For example, as depicted in FIG. 2A, a hydrogel conductor device is provided by encapsulating sinusoidal-shape titanium wires 18 in a hydrogel 12 matrix. This provides a stretchable and robust hydrogel conductor device 10 (e.g., DC conductor). If desired, the hydrogel 12 matrix can be transparent so that the internal components are visible. Using the methods of the present invention, particularly by using chemical silanization treatment, the long-chain polymer network of the tough hydrogel 2 matrix is chemically anchored onto the outer surface 19 of the silanized titanium wires 18 via covalent crosslinks 11. As a result, the wavy titanium wires 18 can be highly stretched together with the hydrogel 12 matrix without fracture or debonding due to the robust adhesion between the titanium wires 18 and the hydrogel 12 matrix.

FIG. 2B demonstrates a hydrogel conductor with A/L=0.23 (A=amplitude and L=wavelength) for the encapsulated sinusoidal-shaped titanium wire in various states of stretch (λ of 1.00, 1.21 and 1.36, where is defined as deformed length over undeformed length). The left images depict the entire hydrogel sheet with the encapsulated wire in various states of stretch. The middle image shows an enlarged view of a bend in the sinusoidal-shaped wire in various states of stretch. It can be seen from these images that, except for the region of the wire, the hydrogel conductor is transparent—clearly showing the background of a white paper. As the hydrogel conductor is deformed to different stretches λ of 1.00, 1.21 and 1.36, the sinusoidal-shaped wire decreases its amplitude and increases its wavelength, enabling high stretchability of the hydrogel conductor. Because the hydrogel is much more compliant than titanium wire (shear modulus 10 kPa vs. 40 GPa) and highly stretchable, it can accommodate the shape change of the titanium wire without fracture or delamination. In each of the right images, the strain is depicted by the variation in color/shading within the hydrogel and around the titanium wire. In the unstretched hydrogel (λ=1.00), the strain is uniform throughout the hydrogel as depicted by the uniform shading along the entire hydrogel. As the hydrogel is stretched (λ=1.21 and 1.36) the bends in the sinusoidal shape begin to straighten out. As can be expected, the strain in those areas of the wire that undergo the greatest extent of shape change is the highest. In particular, when the hydrogel is stretched to different extents, the strain ranges from a level of “high strain” (here, around 0.36 for stretch of 1.21, and around 0.75 for stretch of 1.36) along the outer surface of the wire near the bends (innermost shaded/color) to a level of “low strain” (around 0.24 for stretch of 1.21, and around 0.44 for stretch of 1.36) at a distance away from the outer surface of the wire near the bends (outermost shaded/color). It is noted that the areas of the inner surface of the bends undergo relatively little strain (around 0.10 for stretch of 1.21, and around 0.20 for stretch of 1.36). As shown, the areas of high and low are similar for both stretch of 1.21 and 1.36. However, as can be expected, the absolute values of the strain increases as the amount of stretch increases. In particular, as depicted in FIG. 2C, λₘₐₓ was plotted as a function of A/L to compare experimentally measured maximum stretches in hydrogel conductor devices that contained titanium wires with silanized surfaces and without (“pristine”) silanized surfaces. As demonstrated, the hydrogel 12 with the silanized titanium wires 18 encapsulated therein were stretched to the approximately the theoretical extension limit without detachment between the hydrogel 12 matrix and the titanium wires 18. This was not the case with the pristine wires. Further, the hydrogel conductor device (A/L=0.72, diameter D=0.08 mm) was demonstrated to sustain multiple cycles (i.e., 10,000 cycles) of high stretch (i.e., stretch λ=3 times the undeformed length), while maintaining relatively constant resistance, as depicted in FIG. 2D. In particular, the level of resistance for stretch ranging from 1.0 to over 3.0 remained relatively constant for the first cycle (lowermost line), as well as for 100 cycles (middle line) and for 10,000 cycles (uppermost line, which mainly overlaps with the middle line). This relatively constant level of resistance when undergoing stretch indicated a lack of detachment between the hydrogel matrix and the titanium wires.

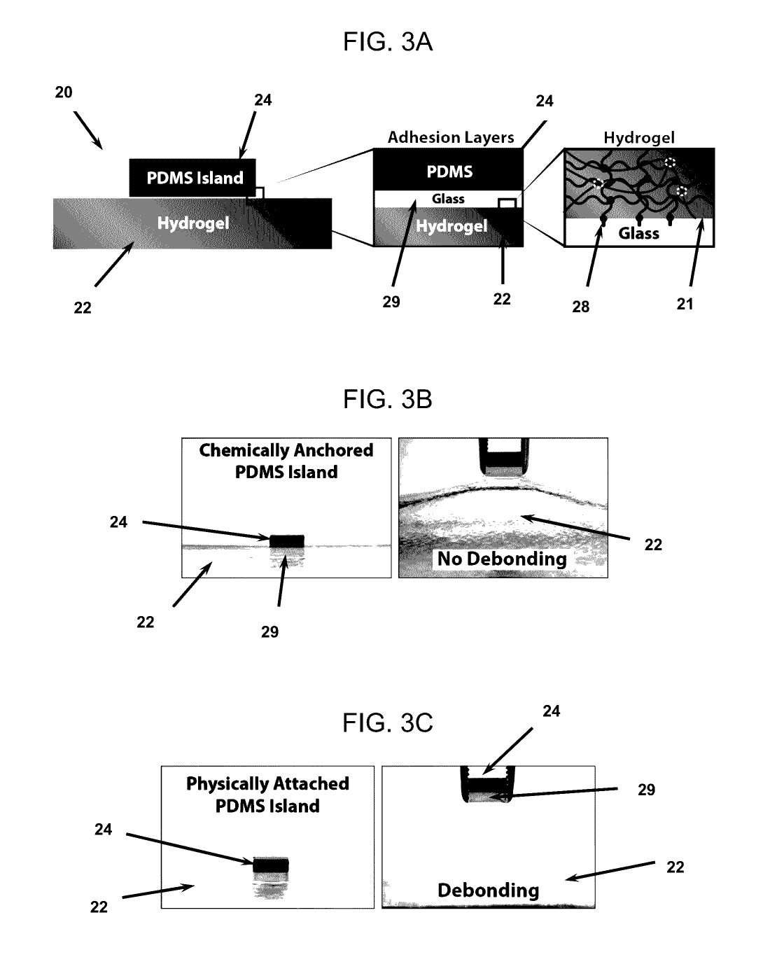

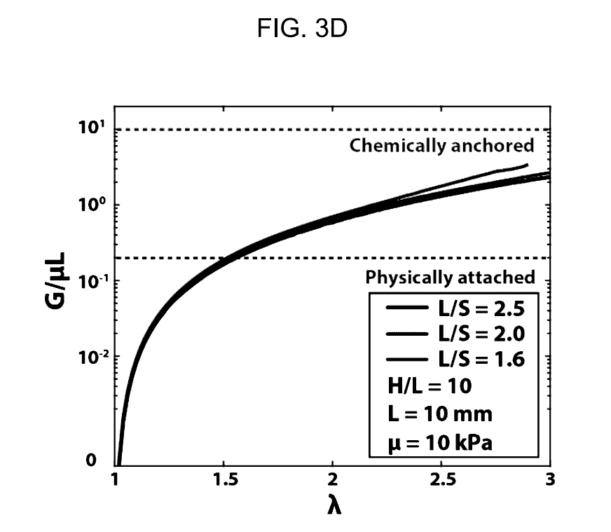

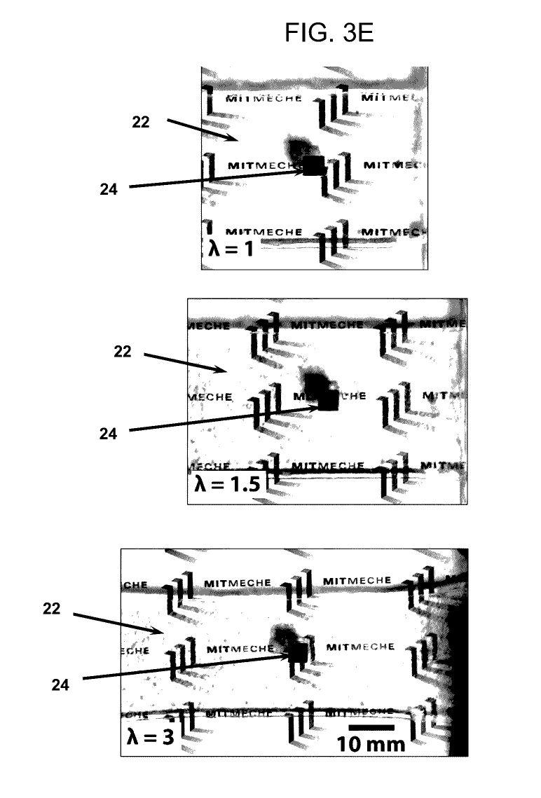

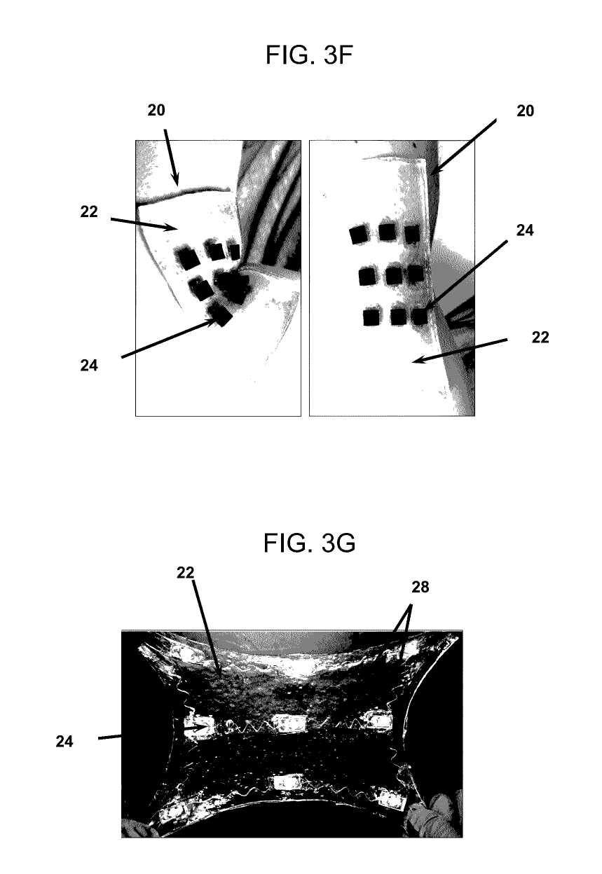

As set out, the present invention further provides a method for encapsulating rigid components within a hydrogel matrix and/or attaching rigid components on a surface of a hydrogel matrix. An exemplary embodiment is shown in FIG. 3A-G, in which rigid electronic components 24 are attached to a surface 21 of a hydrogel 22 matrix. As shown, in order to form tough interfaces between the hydrogel 22 and the rigid electronic components 24, glass slides 29 were used as an intermediate adhesion layers to facilitate in forming tough and stable bonding between the rigid electronic components 24 and the hydrogel 22 matrix. In this embodiment, the rigid electronic components 24 were rigid PDMS (polydimethylsiloxane) chips. According to the present invention, oxygen plasma treated PDMS chips 24 and glass slides 29 were covalently bonded through siloxane bonds, and silanization of the glass slide 29 provided tough covalent bonding 28 with the hydrogel 22.

Consequently, the PDMS chips 24 bonded to the hydrogel 22 via the silanized interface glass slides 29 will not detach from the hydrogel 22 even under states of high deformation. For example, when the PDMS chips were pulled by a tweezer, as demonstrated in FIG. 3B, they did not detach when silanization was carried out. On the other hand, PDMS chips 24 physically bonded (i.e., without silanization) to the hydrogel 22 were easily debonded from hydrogel matrix when pulled by a tweezer, as demonstrated in FIG. 3C. Experimental data is set forth in FIG. 3D, with G/μL plotted as functions of stretch (λ) for varying L/S (with G being the energy releasing rate, μ being the shear modulus of the hydrogel modeled as a neo-Hookean material, L being the width of the chip, and S being the center-to-center distance between two adjacent chips). As shown, L/S of 2.5, 2.0 and 1.6 all follow along generally the same curve, with L/S=1.6 (“blue”) being the highest curve, L/S=2.0 (“red”) falling in the middle, and L/S 2.5 (“black”) being the lowest. As shown, the values of interfacial toughness between the PDMS chips 24 and hydrogels 22 with silanized interfaces (chemically anchored) and without silanized interfaces (physically anchored) were compared. It was found that while chemically anchored interfaces provided interfacial toughness on the order of 1000 J/m², physically anchored interfaces provided significantly less interfacial toughness on the order of about 20 J/m². As depicted in FIG. 3E, a hydrogels 22 having a rigid PDMS chip 24 attached thereto via an intermediate glass slide adhesion layer using silanization treatment was stretched to various states. In the first snapshot, the hydrogel 22 was in an undeformed state (stretch λ=1 times, so the hydrogel length is 1 times its undeformed length). In the second snapshot, the hydrogel is stretched to one and a half times its undeformed length (stretch λ=1.5), and the chemically anchored PDMS chip 24 is still bonded to the hydrogel 22. Even under a high stretch of three times its undeformed state, as depicted in the third snapshot, the chemically anchored PDMS chip 24 is still bonded the hydrogel 22. This strong bonding is due to the robust adhesion between the PDMS chips 24 and the hydrogel 22 via the intermediate glass slide 29 adhesion layer.

In addition, since the present hydrogel devices are soft, wet, and biocompatible, they can be attached to any variety of locations on or within a body. For example, as shown in FIG. 3F, a present invention hydrogel sheet device 20 (thickness ˜1.5 mm) with multiple patterned rigid chips 24 is capable of conformably attaching to a knee, back of knee, an elbow, or similar irregularly shaped surfaces which undergo movement. As illustrated, movement of the body part deforms the hydrogel sheet 30 but does not debond the chips 24.

As depicted in FIG. 3G, both flexible and rigid components were incorporated into a hydrogel matrix. In particular, a hydrogel electronic device was formed of a hydrogel 22 sheet encapsulating an array of rigid LED lights 24 interconnected by stretchable wires 28 (e.g., Ti wires). These stretchable wires 28 and LED lights 24 are incorporated into the hydrogel 22 using the methods set forth herein so as to maintain functionality in both deformed and undeformed states.

As a result, the present invention provides new hydrogel devices which possess numerous advantages over dry polymers. In particular, the hydrogel devices provide stretchable and robust matrices for rigid and flexible components, such as electronic components, and further provide the capability of sustained and controlled drug diffusion. In the current design, drug-delivery channels and reservoirs are patterned in the hydrogel matrix. Drug solutions can then be fed to the channels and reservoirs from external sources. According to some embodiments, the drug solutions can be fed to the channels and reservoirs via controlled flow rate, such as when a temperature detected by one or more of the electronic components reach a particular target range. Thus, controlled convection (i.e., controlling the flow rate of the infused drug solutions) is provided. Thereafter, the one or more drugs diffuse from the reservoirs in a sustained manner based on the properties of the drugs, the hydrogel matrix, the measured temperature and surrounding pressure gradient.

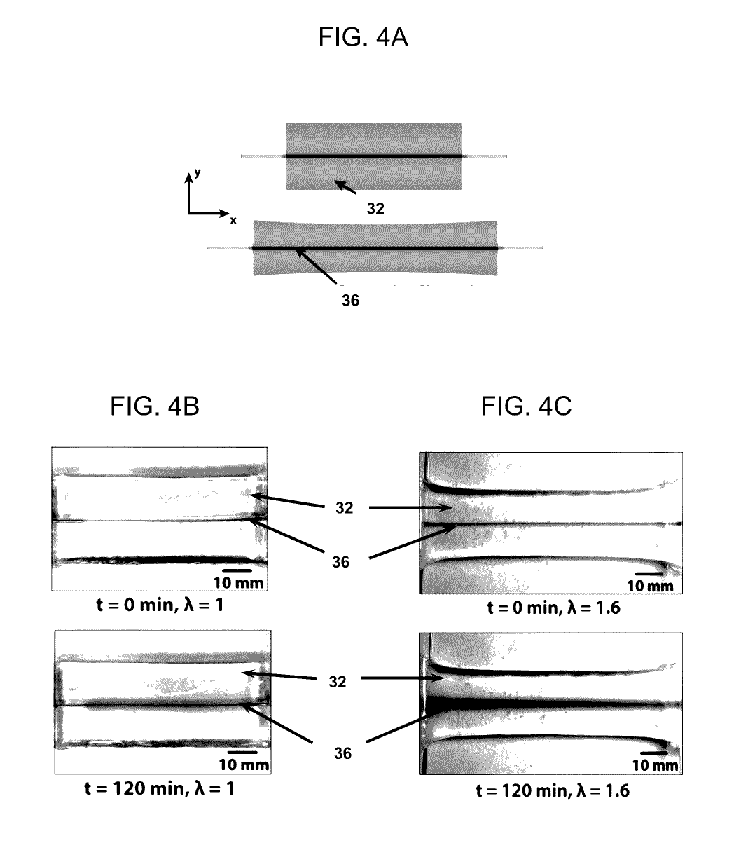

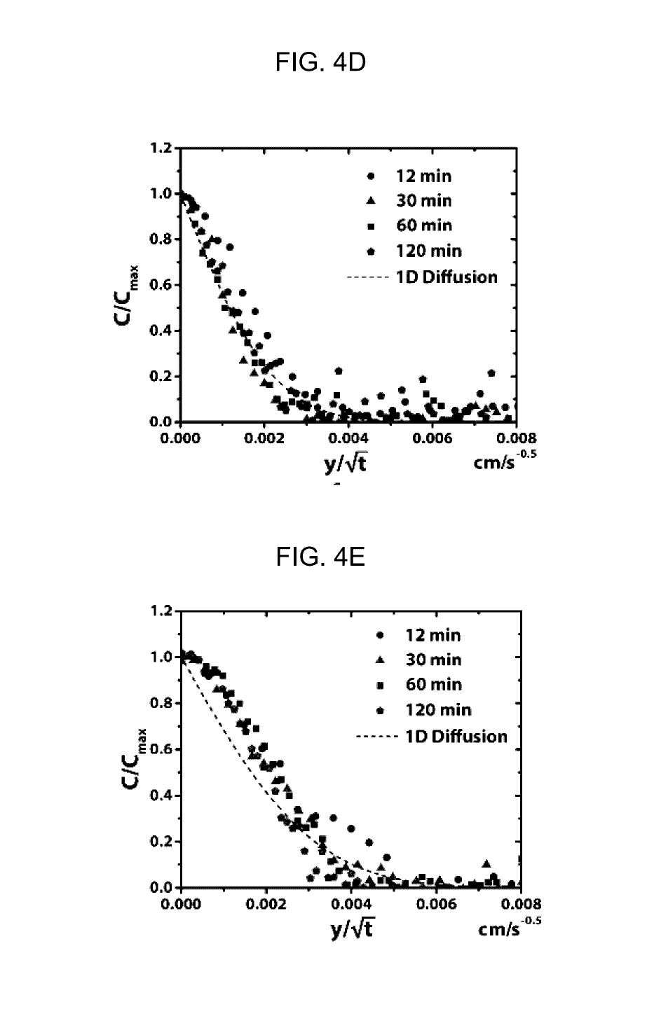

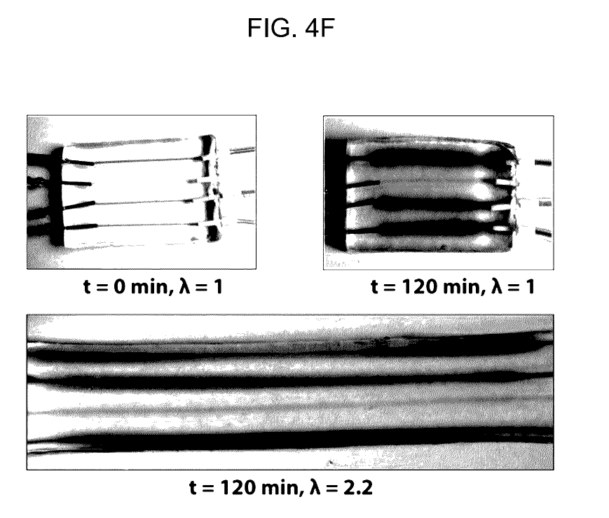

A further advantage of the present devices is that diffusion of the one or more drugs from the reservoirs of the device is relatively constant regardless of the state of device deformation, as schematically illustrated in FIG. 4A. For example, a mock drug comprising a 2% aqueous solution of a red food dye (McCormick®) was fed into a drug-release channel 36 in a hydrogel 32 matrix according to embodiments of the present invention.

FIG. 4B shows experimental snapshots of drug diffusion from an undeformed hydrogel device upon injection of the drug (t=0) and after a time of t=120 minutes had elapsed. FIG. 4C shows experimental snapshots of drug diffusion from the hydrogel device of FIG. 4B, but with a stretch of λ=1.6 times, upon injection of the drug (t=0) and after a time of t=120 minutes has elapsed. In FIGS. 4B-C, for both the undeformed hydrogel and the stretched hydrogel, at t=0 the drug is shown by the solid darkened line running through the middle of the hydrogel within the drug release channel 36. In other words, the drug has just been injected into the channel so it is contained completely within the channel. After a period of time has elapsed at t=120, diffusion of the drug in both the undeformed hydrogel and the stretched hydrogel is shown by the thickened and blurred darkened area which demonstrates that the drug has diffused from drug release channel 36 into the surrounding hydrogel and will continues to do so until it has fully diffused. Similar results are demonstrated in FIG. 4F for a hydrogel containing four drug release channels, with four mock drugs comprising aqueous solutions of different colored food dye (e.g., red, yellow, green and blue) being injected into each of the four channels 36. As demonstrated, diffusion from the undeformed hydrogel device FIG. 4B (as well as the top right illustration in FIG. 4F) and from the stretched hydrogel device FIG. 4C (as well as the bottom illustration in FIG. 4F) was the same or substantially the same, with no noticeable variations. As such, diffusion (i.e., diffusivity or diffusion coefficient) from the hydrogel device in various states of deformation will remain constant or substantially constant regardless of the state of deformation. It is noted that there generally are changes of diffusion coefficient when the material sustains deformation in the actual experimental measurements. However, compared to other transportations, such as convection and migration (which are much faster than diffusion), this change of diffusion coefficient induced by mechanical deformation is small and, thus, is regarded as substantially no change.

As shown in FIGS. 4B-C and 4F, the mock drug diffuses outward from the channel and through the hydrogel matrix into the desired location(s) after a period of time has elapsed. Such diffusion in all directions would be particularly beneficial for implant type devices, wherein diffusion may be desired in all directions relative to the device. On the other hand, if the device is used as a bandage that is adhered to the surface of the skin (or similar devices in which diffusion is only desired in particular directions), the device is configured so as to provide diffusion of the drug in one direction towards the skin (or in one or more particular directions based on the end use of the device) or such that the drug reservoir is disposed near the surface in contact with the skin rather than the surface opposite the skin. Generally, the drug would diffuse into the desired tissue (e.g., a wound) having a wet/moist environment and would not diffuse into the air or in directions other than the desired direction(s). However, if needed, in order to prevent the drug from diffusing in directions other than the desired direction(s), the drug channels/reservoirs could be coated using any material that blocks diffusion of the drug in one or more particular directions. Alternatively, an entire surface or multiple surfaces of the bandage/device can be coated on one or more sides through which diffusion is not desired, using any material that will block diffusion of the drug.

FIGS. 4B and 4D graphically depict the normalized one-dimensional diffusion of the mock drug from inside the undeformed (λ=1) hydrogel channel, while FIGS. 4C and 4E graphically depicts normalized one-dimensional diffusion of mock drug inside the deformed (λ=1.6) hydrogel channel. As shown, the graphical results are substantially the same, demonstrating that diffusion of drugs from the present device did not change significantly regardless of the state of deformation.

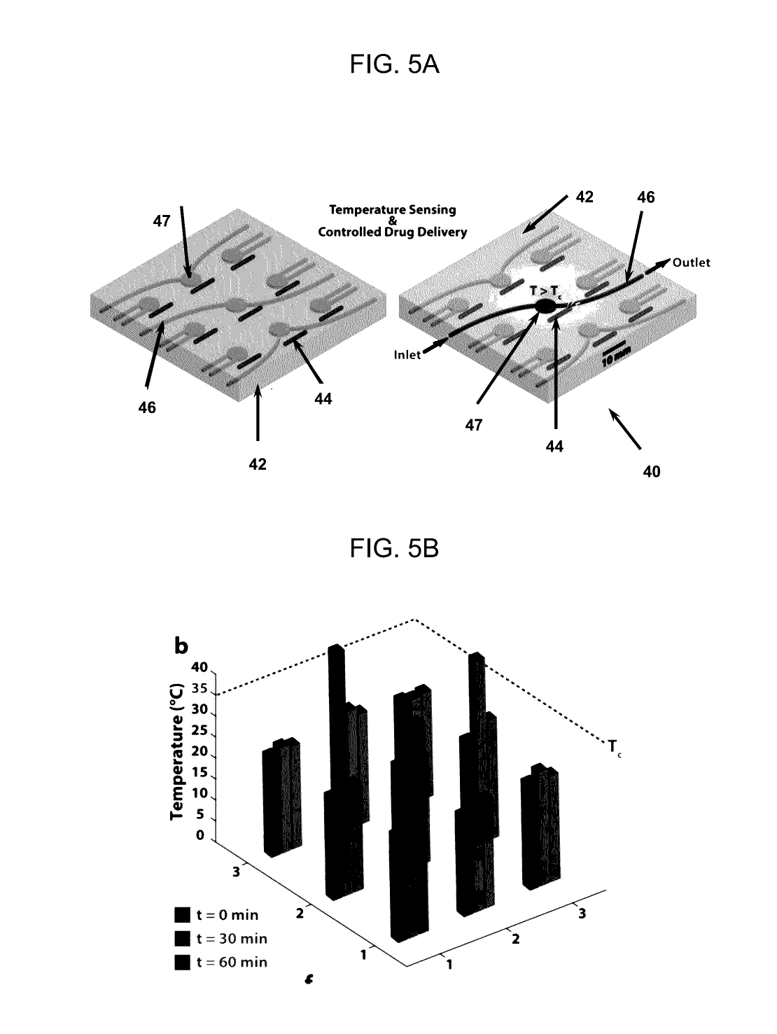

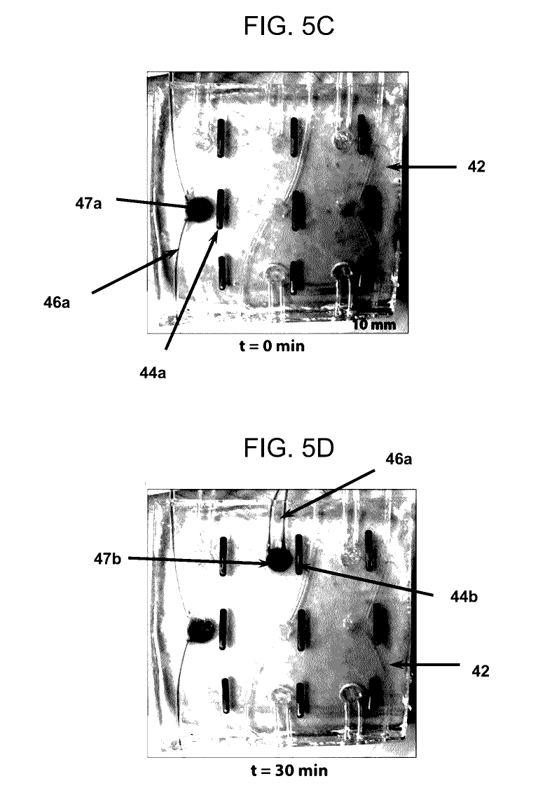

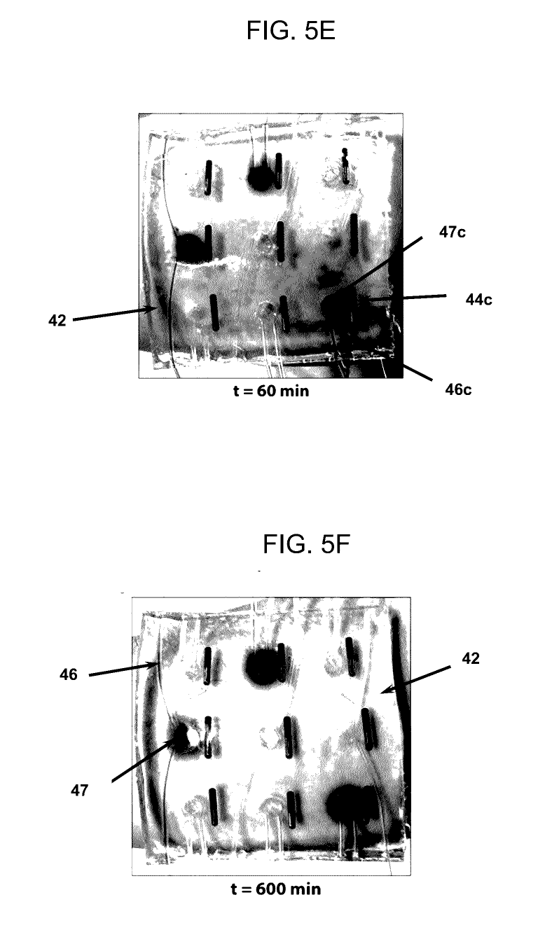

The stretchable and biocompatible hydrogel devices of the present invention can be configured for utilization in a number of applications, including use as a variety of medical devices. For example, as illustrated in FIGS. 5A-F, the present hydrogel device can be configured as a smart wound dressing 40 that combines one or more temperature sensors 44, drug delivery channels 46 and reservoirs 47 patterned into a stretchable and transparent tough hydrogel sheet 42. The smart wound dressing can provide programmable and sustained deliveries of multiple drugs at various locations, such as along various locations of the human skin, based on the temperatures measured at those locations. For example, according to some embodiments as schematically depicted in FIG. 5A, if a temperature sensor 44 detects that the temperature at a location increases above a threshold (T>T_{c}, e.g., T_{c}=35° C.) at a certain time (t=0), a drug solution is delivered through the non-diffusive drug-delivery channel 46 to the corresponding drug reservoir 47. Thereafter, the drug diffuses out of the hydrogel matrix 42 in a controlled and sustained manner. The same procedure can be repeated for other drug delivery channels and reservoirs as the temperature measurements from different temperature sensors 44 over time reach the desired threshold(s).

For example, as shown in FIG. 5C, a first temperature sensor 44a has detected that the temperature at that location reached the threshold temperature and, thus, a drug is delivered to the first drug reservoir 47a corresponding to the first temperature sensor 44a via the first drug delivery channel(s) 46a. As depicted in FIG. 5D, 30 minutes have elapsed subsequent to FIG. 5C, and the drug in the first drug reservoir 47a is diffusing out. In addition, a second temperature sensor 44b has detected that the temperature at that location reached the threshold temperature and, thus, a drug is delivered to the second drug reservoir 47b corresponding to the second temperature sensor 44b via the second drug delivery channel(s) 46b. As depicted in FIG. 5E, 60 minutes have elapsed subsequent to FIG. 5C, and the drugs in the first and second drug reservoirs 47a, 47b are diffusing out. In addition, a third temperature sensor 44c has detected that the temperature at that location reached the threshold temperature and, thus, a drug is delivered to the third drug reservoir 47c corresponding to the third temperature sensor 44c via the third drug delivery channel(s) 46c. Finally, FIG. 5F illustrates the device after 600 minutes have elapsed subsequent to FIG. 5C, and the drugs in the first, second and third drug reservoirs 47a, 47b, 47c are diffusing out.

As such, a smart wound dressing provides programmable and sustained deliveries of different drugs at various locations based on the temperatures measured at those locations. These temperatures can be measured via wireless temperature sensors, and in this embodiment, the channels through which the drugs are delivered to the reservoirs can be non-diffusive channels so that diffusion only occurs from the reservoirs.

According to embodiments of the present invention, programmable delivery and sustained release of one or more drugs is provided. In particular, desired delivery and release of one or more drugs can be achieved by controlling the flow of one or more drug solutions through one or more selected channels and reservoirs in a hydrogel matrix. In addition, the various components disposed within and on the hydrogel matrix are provided in such a way that the components function the same or substantially the same at both undeformed and highly deformed states. By further incorporating a stretchable LED array or similar mechanism within the hydrogel matrix in communication with the drug delivery components, the resulting hydrogel device can be provided in the form of a smart hydrogel wound dressing that (a) is highly deformable, (b) is capable of sensing temperatures at different locations on the skin or other location on or in which it is disposed, and (c) can provide sustained release of one or more drugs to specific locations of the skin or other location on or in which it is disposed based on the sensed temperatures. In addition, according to some embodiments, the hydrogel wound dressing can be transparent so as to allow for visualization and monitoring of the drug delivery.

The present hydrogel devices can be used to provide delivery of drugs in a variety of ways. For example, the hydrogel device can be in the form of a bandage or other type of wound covering to provide transdermal delivery and direct delivery to a wound or damaged skin. In some embodiments, the bandage or wound covering is stretched and subsequently placed and adhered onto a surface. The bandage or wound covering then shrinks back to its unstretched state. As such, the bandage or wound covering could be used to apply compression to an area in need of treatment. According to some embodiments, the hydrogel device is formed as a drug delivery insert and is disposed within the body at a desired location using any known means such as, for example, implanting, injection and ingesting.

What is claimed is:

1. A therapeutic agent delivery system comprising:
a stretchable hydrogel;
at least one therapeutic agent reservoir disposed within the stretchable hydrogel; and
at least one electronic sensor disposed within or on the stretchable hydrogel and in communication with the at least one therapeutic agent reservoir,
wherein a therapeutic agent contained within the at least one therapeutic agent reservoir is delivered through the stretchable hydrogel in response to one or more conditions detected by the at least one electronic sensor component.

2. The system of claim 1, wherein the at least one electronic sensor is in communication with the at least one therapeutic agent reservoir via one or more flexible conductors disposed within the stretchable hydrogel.

3. The system of claim 1 further comprising a therapeutically effective dosage of the therapeutic agent.

4. The system of claim 1, further comprising at least one channel in connection with the at least one reservoir for delivering one or more therapeutic agents into the at least one reservoir.

5. The system of claim 1, wherein the at least one electronic sensor is a temperature sensor, and delivery of therapeutic agent contained within the at least one therapeutic agent reservoir is regulated based upon temperature.

6. The system of claim 1, wherein the stretchable hydrogel has a stiffness of about 10 to 100 kilopascals.

7. The system of claim 1, wherein the stretchable hydrogel is in the form of an ingestible capsule or tablet.

8. The system of claim 1, wherein the stretchable hydrogel is in the form of an implant.

9. A transdermal therapeutic agent delivery system comprising:
a stretchable hydrogel sheet;
at least one therapeutic agent reservoir disposed within the stretchable hydrogel; and
at least one electronic sensor disposed within or on the stretchable hydrogel and in communication with the at least one therapeutic agent reservoir,
wherein delivery of a therapeutic agent contained within the at least one therapeutic agent reservoir is regulated based upon one or more conditions detected by the at least one electronic sensor.

10. The system of claim 9, wherein the at least one electronic sensor is in communication with the at least one therapeutic agent reservoir via one or more flexible conductors disposed within the stretchable hydrogel.

11. The system of claim 9 further comprising a therapeutically effective dosage of the therapeutic agent.

12. The system of claim 9, further comprising at least one channel in connection with the at least one reservoir for delivering one or more therapeutic agents into the at least one reservoir.

13. The system of claim 9, wherein the at least one electronic sensor is a temperature sensor, and delivery of therapeutic agent contained within the at least one therapeutic agent reservoir is regulated based upon temperature.

14. The system of claim 9, wherein the stretchable hydrogel has a stiffness of about 10 to 100 kilopascals.

15. The system of claim 9, wherein the stretchable hydrogel sheet is configured to adhere and conform to a surface on which it is placed.

16. A method for transdermal delivery of one or more therapeutic agents comprising:
providing a stretchable hydrogel sheet, the stretchable hydrogel sheet having at least one therapeutic agent reservoir disposed therein, at least one fluid delivery channel in connection with the at least one therapeutic agent reservoir, and at least one electronic sensor disposed within or on the stretchable hydrogel and in communication with the at least one therapeutic agent reservoir;
disposing the stretchable hydrogel sheet on a surface of a subject's skin;
delivering at least one therapeutic agent to the at least one therapeutic agent reservoir via the at least one fluid delivery channel;
sensing one or more conditions of the subject using the electronic sensor; and
automatically regulating delivery of the at least one therapeutic agent contained within the at least one therapeutic agent reservoir through the hydrogel sheet and to the subject based upon one or more conditions.

17. The method of claim 16, further comprising, prior to disposing the stretchable hydrogel sheet on the surface of the subject's skin, stretching the hydrogel sheet and disposing the stretched hydrogel sheet on the surface of the subject's skin.

18. The method of claim 16, wherein the hydrogel sheet is disposed on a wounded or burned surface of the subject's skin.

19. The method of claim 16 wherein at least one condition of the subject is temperature, and wherein therapeutic agent is delivered to the subject upon an increase in temperature above a threshold level.

20. A method for delivery of one or more therapeutic agents to a subject comprising:
providing a stretchable hydrogel, the stretchable hydrogel having at least one therapeutic agent reservoir disposed therein, at least one therapeutic agent contained within the at least one therapeutic agent reservoir, and at least one electronic sensor disposed within or on the stretchable hydrogel and in communication with the at least one therapeutic agent reservoir;
delivering the stretchable hydrogel to a subject internally;
sensing one or more conditions of the subject using the electronic sensor; and
automatically regulating delivery of the at least one therapeutic agent contained within the at least one therapeutic agent reservoir through the hydrogel and to the subject based upon one or more conditions.

21. The method of claim 20, wherein delivering comprises implanting.

22. The method of claim 20, wherein delivering comprises ingesting.
